(19) United States
(12) Patent Application Publication
(10) Pub. No.: US 2009/0254111 A1
(43) Pub. Date: Oct. 8, 2009

(54) DEVICE FOR IMPLANTING OCCLUSION SPIRALS COMPRISING AN INTERIOR SECURING ELEMENT

(76) Inventors: Hermann Monstadt, Bochum (DE); Ralph Bodenburg, Bochum (DE)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/912,901

(22) PCT Filed: Apr. 28, 2006
(86) PCT No.: PCT/EP2006/004000

§ 371 Date: Aug. 12, 2008

(30) Foreign Application Priority Data
Apr. 28, 2005 (DE) 10 2005 019 782.5

(51) Int. Cl. A61M 29/00

(52) U.S. Cl. 606/191

(57) ABSTRACT

The invention relates to a device for the implantation of occlusion helixes (3) into body cavities or blood vessels, in particular aneurysms (12), with at least one occlusion helix (3) comprising a plurality of windings and being movably arranged in longitudinal direction within a catheter (1), and one securing means (9) passing at least partially through the lumen of the occlusion helix (3), with said securing means (9) being fixed in its end areas inside the occlusion helix (3) and consisting of at least two wires with each of the wires having a diameter of less than 0.02 mm. The use of a plurality of wires of relatively small diameter for the securing means (9) enables high flexibility and at the same time high tensile strength properties to be attained. In this way, two opposed characteristics can be achieved which at first glance appear to be mutually exclusive.

The invention relates to a device for the implantation of occlusion helixes into body cavities or blood vessels, in particular aneurysms, with at least one occlusion helix comprising wires that form a plurality of windings and being movably arranged in longitudinal direction within a catheter, and one securing means passing at least partially through the lumen of the occlusion helix, with said securing means being fixed in its end areas inside the occlusion helix.

The use of endovascular techniques for the occlusion of body cavities or vessels such as arteries, veins, fallopian tubes or vascular deformities (for example, vascular aneurysms) is known in the art. In this case, the occlusion helix is usually introduced by means of an endovascular insertion wire through a catheter into the cavity to be occluded and deposited therein.

Before placement may commence the occlusion helixes are maneuvered with the help of the catheter through the blood vessel system and, at the target site, advanced out of the catheter and into the cavity to be occluded. Ideally, the separation/severance of the helix follows these steps. In the event of a wrong placement of the occlusion helix or if too large an occlusion helix has been selected for the area to be occluded said helix must then be repositioned or completely retracted into the catheter to subsequently enable such an occlusion helix to be correctly positioned or a correctly sized helix to be placed in position. Maneuvers of this kind involve risks in that parts of the helix are pulled apart and elongate due to the tensile or torsional stresses applied and in this way become elastically deformed irreversibly, are torn off or broken which may give rise to life-threatening embolism.

To minimize this danger it has been known, inter alia from publication WO 99/09894 A1, to securely provide inside the occlusion helix a flexible securing means consisting of a polymer material. With the help of such a securing means it is ensured that a wrongly positioned occlusion helix can be safely retracted without the risk of portions of the occlusion helix being pulled apart so that hazards as mentioned above can be minimized in this manner.

Aside from polymeric securing means of this nature further securing means are known that are made of metals having shape-memory properties, for example nitinol. Such securing means are disclosed in WO 2004/014239 A1. When compared to polymer materials significant advantages, especially with regard to tensile strength, can be achieved if shape-memory metals are used because even in the event of high tensile loads being exerted the securing means is not expected to break.

On the other hand, using a securing means made of metal will result in a relatively stiff occlusion helix to be moved forward via the catheter, said stiffness being due to the relative high bending strength of the wire forming the securing means. However, if the stiffness of the occlusion helix exceeds a certain limit both advancing the occlusion helix through narrow blood vessels and adapting it to the interior of an aneurysm into which the occlusion helix is inserted will be impeded. Since not only flexibility but at the same time a high bending strength of the securing means are desirable factors to be aimed at in this context a compromise was always needed here. While using a polymer material warrants high flexibility this material often does not have sufficient tensile strength, with this situation being almost exactly vice versa when a metal wire is employed. The same applies to the size of the securing means; while a wire of larger diameter has an adequately high tensile strength it suffers at the same time disadvantages with respect to flexibility whereas a thinner wire is sufficiently flexible but does not yield the tensile strength needed. Therefore, as described in WO 2004/014239 A1, wires having a diameter ranging between 0.03 and 0.1 mm are preferably used.

Proceeding from what is known from prior art as described above it is therefore the objective of the invention to provide a device of the kind first mentioned above being fitted with a securing means that not only has adequate flexibility but at the same time also sufficient tensile strength properties.

According to the invention this objective is achieved by providing a device for the implantation of occlusion helixes into body cavities or blood vessels, in particular aneurysms, with at least one occlusion helix comprising wires that form a plurality of windings and being movably arranged in longitudinal direction within a catheter, and one securing means passing at least partially through the lumen of the occlusion helix, with said securing means being fixed in its end areas inside the occlusion helix and consisting of at least two wires with each of the wires having a diameter of less than 0.02 mm.

The invention is based on findings according to which the tensile strength in fact increases proportionately to the cross-sectional area of the wires used as securing means but the flexibility of the securing means altogether increases significantly and disproportionately when the diameter of the wires decreases. Accordingly, a securing means comprising two wires of a given cross-sectional area for example has the same tensile strength as a securing means comprising one wire of identical cross-sectional area, however the flexibility of the securing means will increase significantly.

Theoretically, the phenomenon can be elucidated as follows:

The calculation of the possible flexure f is based on the following formula:

$$f = \frac{\frac{F}{n} * {l}^{3}}{3 * E * {l}_{D}}$$

where:

### F=Force

E=Modulus of elasticity
n=Number of wires
l=Length
l_{D}=Axial resistance moment;

The axial moment of resistance l_{D} is determined acc. to formula

$${l}_{D} = \frac{π * {D}^{4}}{64}$$

with D being the diameter of the wire or wires used as securing means. As is easily seen the diameter influences the determined flexure f to the fourth power with said flexure being arrived at if a certain force F is allowed to act on a segment of the securing means. For that reason, flexure f is thus a measure determining the flexibility of the securing means. On the other hand, the number n of the wires used enter the calculation of flexure f only singly so that several thin wires can be considerably more flexible than one thicker wire.

As already mentioned above, the tensile strength of the securing means is directly proportionate to the overall cross-sectional area A, with A being determined as per

$$A = \frac{n * π * {D}^{2}}{4}$$

In this case the diameter of the individual wires takes effect only quadratically.

The following table compares various flexibility and tensile strength values that have been determined:

| NiTi Wire | 1 × Ø 0.024 mm | 2 × Ø 0.018 mm | 3 × Ø 0.018 mm | 3 × Ø 0.015 mm |
|---|---|---|---|---|
| Flexure | 100% | 158% | 104% | 218% |
| (Flexibility) | | | | |
| Tensile strength | 100% | 112% | 169% | 117% |

It is thus evident from the above that flexibility increases by more than factor two and even the tensile strength properties slightly improve if, for example, three wires each having a diameter of 0.015 mm are used instead of one wire 0.024 mm in diameter, which in securing means provided according to the known state of the art are two opposed characteristics that in fact should exclude each other. Moreover, wires can be employed for the manufacture of the securing means that otherwise would not prove useful as their diameters are too small to yield the required tensile strength.

According to the invention two to four wires are preferably used for the securing means, and using three wires according to the above table has turned out to be of particular advantage. Basically, although it would be feasible to employ more than four wires of even smaller diameter the relevant manufacturing expenditure would increase if a greater number of wires is used with their diameter being further reduced so that compromising on using two to four wires is considered to be reasonable and beneficial.

In particular, the wires may be made of a metal having shape-memory properties. As regards the shape-memory properties of the securing means these may be due to a thermal or mechanical shape-memory effect. Especially proven materials used for this purpose are titanium-nickel alloys, in particular an alloy known to those skilled in the art under the name of nitinol. Furthermore, alternative materials may be used as well, for example iron or copper based alloys. The properties of a shape-memory material can be precisely controlled or influenced by a person skilled in the art in a known manner by selecting exactly the material composition required.

In particular, the wires forming part of the securing means may consist of dissimilar materials to enable the different material properties to be combined in this manner. Furthermore, the wires of the securing means may also be subjected to different treatment methods to provide them with different properties, for example especially high flexibility on the one hand and a particularly high tensile strength on the other. Another possibility is to manufacture one or several wires of materials having shape-memory properties and one or several wires of other materials that add other properties.

The securing means made of a shape-memory material may also be preformed into a superimposed structure which it assumes when it is released from the catheter used for the placement of an occlusion helix. In this way, a desired superimposed structure can be imposed on the occlusion helix which it assumes after being released from the catheter and placed, for example, in an aneurysm. The formation of such a secondary structure, for instance of helical coils or basket shape, is of advantage basically, because in this way the aneurysm is filled out particularly well to make sure an effective thrombozation of the aneurysm can be achieved. If thought expedient, the occlusion helix itself may be preformed into such a superimposed structure which it assumes when it is released from the catheter. However, it may nevertheless be sufficient to exclusively preform the securing means and not the occlusion helix itself, provided the force exerted by the securing means is great enough to also force the occlusion helix into the shape predetermined by the securing means. The force exerted by the securing means is brought about due to the securing means being liberated and released from the constraint of the surrounding catheter when placed into an aneurysm and retransformed returning to its austenite phase which causes it to assume the previously impressed structure. Additionally or alternatively, a temperature induced transformation may also be caused when the securing means, upon being released from the catheter, is subjected to the elevated temperature prevailing in the blood stream.

Preferably, the wires forming the securing means run parallel to each other because in this case the beneficial effect described hereinbefore is produced automatically. Nevertheless, the wires may also be twisted around each other or braided which on the one hand makes it easier to handle the securing means as a unitary object, its flexibility, however, will decrease.

Expediently, the occlusion helix is designed to have one or several electrolytically corrodible locations. The method of electrolytic severance of occlusion helixes is sufficiently known to competent persons skilled in the art and offers many advantages in terms of practicability, safety, speed and cost-effectiveness over other techniques known from prior art and aimed at separating occlusion helixes. For such an electrolytic severance an electrically isolating catheter and a voltage source is used, with the occlusion helix itself serving as anode. A cathode is usually positioned on the body surface. Aside from the provision of severance elements in the occlusion helix serving as electrolytically corrodible locations, also prior-art devices are known which have detachment points arranged in the guide wire.

As is known from DE 100 10 840 A1 it is considered particularly expedient if the occlusion helix has several electrolytically corrodible locations, with a securing means being arranged in each segment of the occlusion helix situated between these locations. This, preferably, extends from one end to the other end of each segment. This embodiment enables variably sized lengths of occlusion helixes to be placed so that helixes of exactly the right length can be positioned in the aneurysm. If necessary, even several sections of the same occlusion helix may be separated one after the other and introduced into the cavity to be occluded. This is beneficial not only in terms of costs and time but also serves to further minimize surgery risks. Also, the application of this method dispenses with the need to always keep ready and use differently sized occlusion helixes for placement into aneurysms of different size but instead enables a uniformly sized device to be employed and, as required in each individual case, differently sized sections of the occlusion helix to be placed into the aneurysm.

If necessary, several spaced occlusion helixes may also be employed, with one electrolytically corrodible severance element each being arranged between the individual occlusion helixes. In this case, one securing means should be provided in each individual occlusion helix.

The application of occlusion helixes having a plurality of electrolytically corridible locations is based on findings according to which the specific severance location of the occlusion helix that is situated nearest to the distal end of the catheter is separated by electrolysis when a current is applied to such a device. This is due to the fact that on the one hand the electrolytically corrodible locations in the catheter are isolated from the ionic medium through the catheter and thus not affected by electrolysis and, on the other hand, the current density decreases from proximal to distal owing to the distally increasing resistance in the occlusion helix or helixes. As a result of this, the electrolytically corrodible point which, viewed in distal direction, is closest to the distal end of the catheter is subjected to the most intensive electrolytic process and is thus preferentially dissolved. Arranging one securing means each in the segments of the occlusion helix between the electrolytically corrodible locations or inside the individual occlusion helixes serves the purpose of safeguarding each individual segment so that a maximum degree of safety is achieved with respect to preventing the occlusion helix from being torn off.

The wires forming the securing means may be enwrapped by an insulating sheathing or coating. This is particularly expedient if a device is employed that has electrolytically corrodible severance locations since such an insulating sheathing or coating will prevent the securing means proper from being attacked by electrolytic corrosion. Moreover, providing insulating means will result in the current density at the severance element being as high as possible to enable the separation/severance action to take place quickly.

Expediently, the securing means extends from the proximal to the distal end of the occlusion helix or at least the separable segment of the occlusion helix to make sure the entire occlusion helix is safe against being torn off. It is, moreover, viewed expedient for the securing means to extend up to the distal tip section of the occlusion helix which is subjected to particularly high stresses when placed into the blood vessel and for this reason usually has a rounded shape to avoid wall ruptures.

Basically, the securing means may be attached with its two ends directly or indirectly to the occlusion helix. For an indirect attachment the securing means is fixed inside the occlusion helix with the help of transition elements connected to the securing means and the occlusion helix. This fixing method may, for example, be a crimping connection and, inter alia, microcoils may be used as transition elements the outer diameter of which is matched to the inside diameter of the occlusion helix with said microcoils being at least partially inserted into the occlusion helix. This embodiment is especially cost-effective because it can be manufactured using customary occlusion helixes into which the combination of securing means and at least two microcoils attached to the end of the securing means is inserted and connected to the occlusion helix by means of established processes. To connect the securing means to the transition element (microcoil) and transition element to the occlusion helix methods sufficiently known to persons skilled in the art are suited such as welding, soldering, bonding or mechanical (i.e. force- and/or form-closed) joining processes.

Aside from this, it is also feasible to attach the securing means at least in one end area to the occlusion helix or the transition element by means of a frictional connection. As is doubtlessly possible without difficulty for persons skilled in the art, the frictional connection can be precisely designed in such a manner that the frictional forces holding the securing means are lower than the pull force that must act on the securing means in order to bring about its failure or breakage. On the other hand, the frictional force must be set high enough to enable a retraction and repositioning of the occlusion helix to be performed under normal conditions without problems. In case the tensile force increases to such an extent that the securing means must be expected to break, said securing means is released at its point of attachment within the microcoil and pulls out of the same so that the frictional connection becomes detached and a failure/breakage of the securing means is avoided. Moreover, the frictional connection will not become detached abruptly as in the case of a failure of the securing means but gradually so that no sudden forces are exerted and permitted to cause negative effects in the blood vessel.

Preferably, the securing means of the device according to the invention is a little longer than the particular portion of the occlusion helix through which lumen it extends. The length of the securing means established in this manner makes sure that in the absence of external forces being exerted the securing means in the occlusion helix is not subjected to tensile stresses and, moreover, the flexibility of the occlusion helix is restricted even less.

Aside from the above described measures aimed at increasing the flexibility of the occlusion helix also the diameter of the wire used to form the occlusion helix proper may be selected smaller than is customarily the case. Accordingly, a wire may be employed that has a diameter of only 0.03 mm so that, as has been found, the flexibility will also improve. The occlusion helixes are expediently made of platinum or platinum alloys which have proven their worth. Especially preferred here is the use of platinum-iridium alloys. Alloys of this kind feature a high radiopaqueness and in this way enable the occlusion helix in the body to be visualized.

The severance elements designed to be quickly corrodible preferably consist of alloyed steel. Preferred in this context is stainless steel material having a chromium content of between 12 and 20% w/w. If thought feasible these severance elements may be pre-corroded, for example by a heat treatment, which causes the metal structure to be modified such that it very quickly disintegrates in an electrolyte when an electric voltage is applied. Another possibility to design the severance elements so as to be highly corrodible is to make use of material combinations for the relevant areas that are conducive to the formation of local elements. Examples in this case are combinations of stainless steels with noble metals or noble metal alloys, in particular platinum alloys.

Expediently, an insertion aid in the form of a guide wire is attached proximally to the occlusion helix. Guide wires of this kind have proven their worth in aiding occlusion helixes to be passed through a catheter towards a cavity to be occluded.

The device according to the invention may also be provided directly in combination with a catheter, especially a microcatheter, through which the occlusion helix can be moved forward towards the body cavity or blood vessel to be occluded. The catheter used and the employed occlusion helix in this case shall be matched with respect to their size. If necessary, the catheter also may exert constraint on the occlusion helix and on the securing means resulting in the occlusion helix to assume in the aneurysm its or the securing means' previously impressed secondary structure not earlier than after it has been liberated and released from such constraint. Expediently, the catheter is also provided with radiopaque markers enabling a placement in the target area with the aid of known imaging methods.

The inventive device is preferably intended for use in veterinary or human medicine and, more particularly, for the endovascular treatment of intracranial aneurysms and acquired or innate arteriovenous blood vessel malformations and/or fistulas or for the embolization of tumors by thrombozation.

Further elucidation of the invention is provided by way of examples through the enclosed figures, where

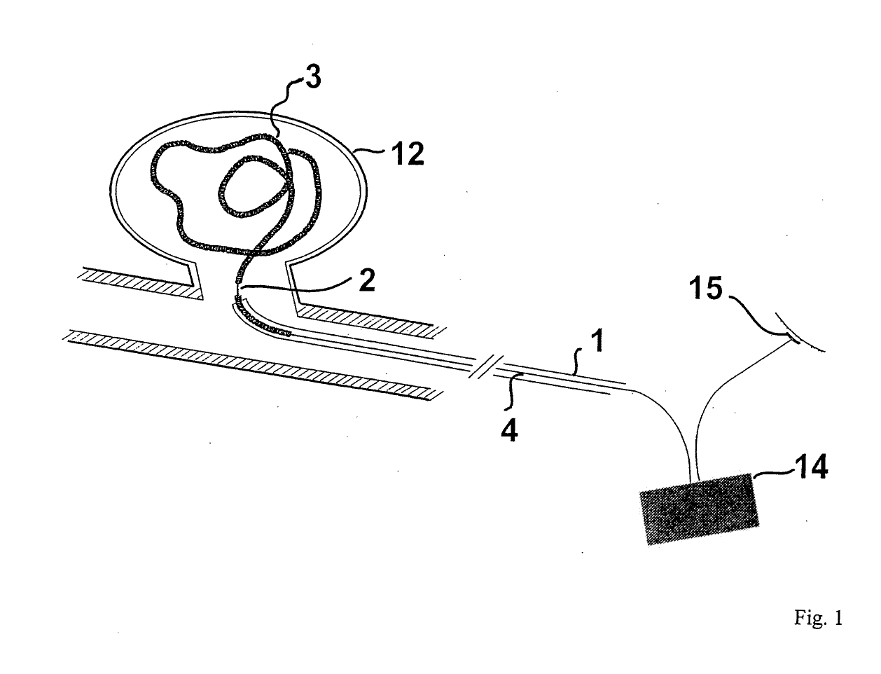

FIG. 1 is a schematic representation showing the positioning of an occlusion helix in an aciniform aneurysm with the help of the inventive device;

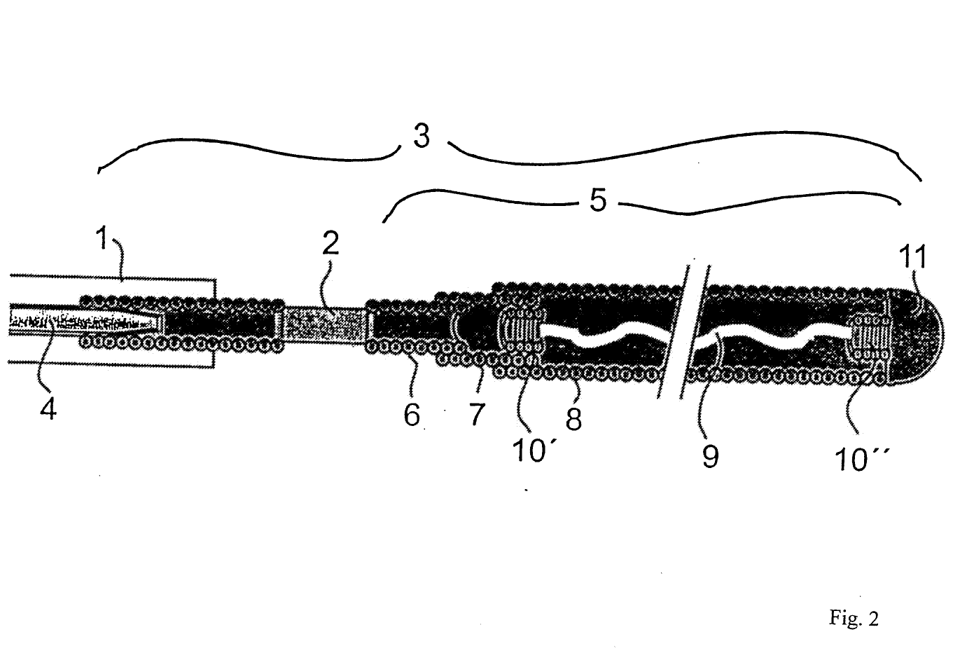

FIG. 2 is a longitudinal section of an inventive device illustrated as a side view and

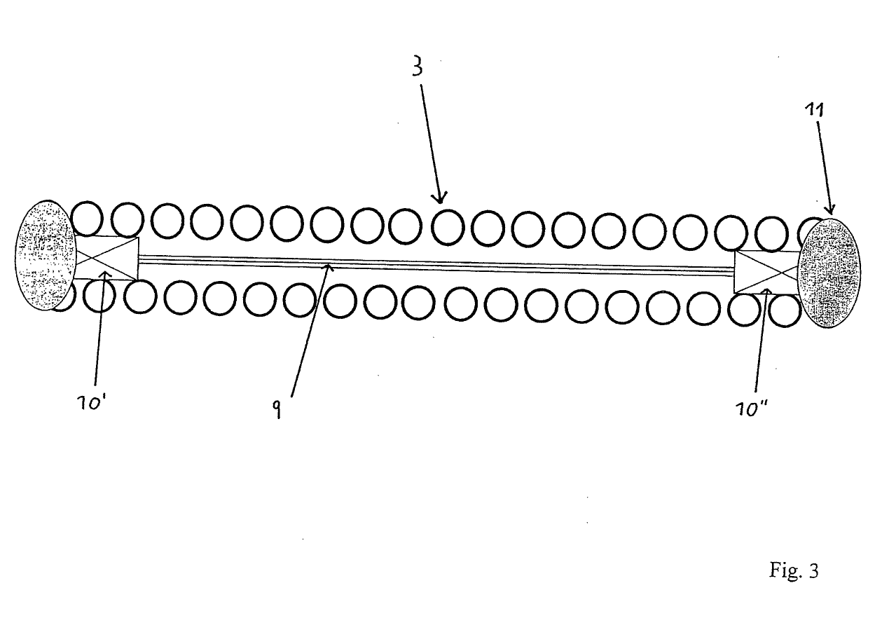

FIG. 3 shows part of the device according to the invention including the securing means and shown as a side view.

FIG. 1 shows a view of the occlusion helix 3 placed into an aciniform aneurysm 12. The occlusion helix 3 is moved distally within catheter 1 with the help of the guide wire 4. When correctly positioned the occlusion helix 3 exits from the end of catheter 1 and is introduced into and fills up the cavity formed through the aciniform aneurysm 12. Within the aneurysm 12 the occlusion helix 3 forms secondary coils or turns which in particular can be caused by a stress- and/or temperature-induced transformation from the martensitic to the austenitic phase of the occlusion helix 3 and/or securing means, which has not been shown here, inside the occlusion helix 3. Due to the formation of secondary coils or turns the aneurysm 12 is filled up particularly effectively.

As soon as a certain length of the occlusion helix 3, which suits the volume of the cavity to be filled, has been placed into the aneurysm 12 the electrolytic separation is effected at the electrolytically corrodible location 2. For this purpose, an electric voltage coming from voltage source 14 is applied to the location 2 with the electrolytically corrodible location 2 (severance element) serving as anode. The cathode 15 is positioned on the body surface. As per a preferred embodiment several severance elements 2 are provided within the area of the occlusion helix 3 so that the length of the introduced occlusion helix 3 can be appropriately sized to suit the respective aneurysm 12. Separation/severance usually takes place within a short time of 1 minute or less (at 2 V, 2 mA).

FIG. 2 shows the inventive device as an amplified view. The occlusion helix 3 made of a platinum-iridium alloy and provided with an electrolytically corrodible location 2 of stainless steel is moved with the aid of the guide wire 4, which is attached to the occlusion helix 3 by a welding method, through the electrically insulating catheter 1 out of the micro-catheter and in the blood vessel system, with catheter 1 in fact being a flexibly designed micro-catheter. The occlusion helix 3 is provided with an electrolytically separable segment 5 which is connected by means of welding using non-matching filler metal to the electrolytically corrodible location 2 arranged proximally of it. At its proximal end the segment 5 is provided with a first microcoil 6 of small diameter which at its proximal end is attached by welding to the adjacent electrolytically corrodible location 2 and at its distal end to another microcoil 7 of medium diameter. This microcoil 7 of medium diameter embraces partially the first microcoil 6 to which it is also connected by means of a welding method. The second microcoil 7 is then surrounded by the third microcoil 8 having the greatest diameter and accommodating the securing means 9 which extends through its interior and consists of a nickel-titanium alloy. It is to be noted, however, that occlusion helix 3 illustrated here is just a design example so that it is understood that other configurations by means of which the occlusion helix 3 may be connected to the electrolytically corrodible location 2 are feasible as well.

The securing means 9 shown here as a unitary object although it consists of several wires is fixed at its two ends inside the occlusion helix with the help of transition elements 10′, 10″. In this case the transition elements 10′, 10″ as well are designed to form microcoils and are each permanently welded to the adjacent microcoils of the occlusion helix 3. The distal tip 11 of the occlusion helix 3 has been rounded with a view to minimizing aneurysm traumatizing risks or wall ruptures. Inside, the tip 11 is permanently attached by welding methods to microcoil 10″ arranged distally and serving as connecting element so that should the tip 11 or adjacent segments of the occlusion helix 3 break off or be torn off the tip 11 cannot enter the blood stream where it could cause embolism hazards.

Finally, FIG. 3 is a greatly simplified representation of the occlusion helix 3 accommodating the securing means 9 according to the invention. As can be seen the securing means 9 consists of a total of three wires extending parallelly to each other and being connected to the proximal and distal end each of the occlusion helix 3 via transition elements 10′, 10″, in this case designed as a pressed (or crimped) microcoil each. Through the use of several wires of which each has a diameter smaller than that of the wires known from prior art used for the manufacture of a securing means it is ensured that the occlusion helix 3 has highly flexible properties and the securing means 9 still possesses sufficient tensile strength.

1. Device for the implantation of occlusion helixes (3) into body cavities or blood vessels, in particular aneurysms (12), with at least one occlusion helix (3) comprising a plurality of windings and being movably arranged in longitudinal direction within a catheter (1), and one securing means (9) passing at least partially through the lumen of the occlusion helix (3), with said securing means (9) being fixed in its end areas inside the occlusion helix (3) characterized in that the securing means (9) consists of at least two wires with each of the wires having a diameter of less than 0.02 mm.

2. The device according to claim 1, characterized in that the securing means (9) consists of two to four wires.

3. Device according to claim 1 or 2, characterized in that the wires are made of a metal having shape-memory properties.

4. Device according to claim 3, characterized in that the wires consist of a nickel-titanium alloy, in particular of nitinol.

5. Device according to claim 3 or 4, characterized in that the securing means (9) has been preformed into a superimposed structure which it assumes when it is released from a catheter (1) used for the placement of the occlusion helix (3).

6. Device according to any one of the claims 1 to 5, characterized in that the wires extend parallelly.

7. Device according to any one of the claims 1 to 5, characterized in that the wires are twisted around each other or braided.

8. Device according to any one of the claims 1 to 7, characterized in that the occlusion helix (3) has been provided with one or several electrolytically corrodible locations (2).

9. The device according to claim 8, characterized in that the occlusion helix (3) has several spaced locations (2) that are electrolytically corrodible, and a securing means (9) is arranged between these locations (2) in each segment of the occlusion helix (3).

10. Device according to any one of claims 1 to 7, characterized by several spaced occlusion helixes (3), with one electrolytically corrodible severance element (2) each being arranged between the individual occlusion helixes (3).

11. The device according to claim 10, characterized in that a securing means (9) each is arranged in the individual occlusion helixes (3).

12. Device according to any one of the claims 1 to 11, characterized in that the wires are embraced by an electrically insulating sheathing or coating.

13. Device according to any one of the claims 1 to 12, characterized in that the securing means (9) extends from the proximal to the distal end of the occlusion helix (3) or the separable segment (5) of the occlusion helix (3).

14. Device according to any one of the claims 1 to 13, characterized in that the securing means (9) is directly attached at one or both of its ends to the occlusion helix (3).

15. Device according to any one of the claims 1 to 13, characterized in that the securing means (9) at one or both of its ends is fixed via transition elements (10′, 10″) inside the occlusion helix (3), said elements being connected with the securing means (9) and the occlusion helix (3).

16. Device according to claim 14 or 15, characterized in that the securing means (9) is attached at least in one end area to the occlusion helix (3) or a transition element (10′, 10″) by means of a frictional connection.

17. Device according to any one of the claims 1 to 16, characterized in that an insertion aid in the form of a guide wire (4) is arranged proximally to the occlusion helix (3).

18. Device according to any one of the claims 1 to 17, characterized in that the device is provided in combination with a catheter (1) through which the occlusion helix (3) can be moved forward towards the body cavity or blood vessel to be occluded.
